(19) United States
(12) Patent Application Publication
(10) Pub. No.: US 2004/0143132 A1
(43) Pub. Date: Jul. 22, 2004

(54) Regeneration of catalyst for use in production of lower aliphatic carboxylic acid ester and production of lower aliphatic carboxylic acid ester

(76) Inventors: Etsuko Kadowaki, Oita (JP); Kyoichi Watanabe, Oita (JP); Yumiko Obata, Oita (JP); Hiroshi Uchida, Oita (JP)

Correspondence Address: SUGHRUE MION, PLLC, 2100 PENNSYLVANIA AVENUE, N.W., SUITE 800, WASHINGTON, DC, 20037, US

(21) Appl. No.: 10/477,922

(22) PCT Filed: Jun. 3, 2002
(86) PCT No.: PCT/JP02/05445

(30) Foreign Application Priority Data
Jun. 19, 2001 (JP) 2001-184526

(51) Int. Cl. B01J 38/02; C07C 67/04

(52) U.S. Cl. 560/241; 502/56

(57) ABSTRACT

A catalyst for use in the production of a lower aliphatic carboxylic acid ester by reacting a lower olefin and a lower aliphatic acid ester in a gas phase, which catalyst has been decreased in activity through use, is regenerated by heating to be recovered in activity. The regenerated catalyst is reused for the production of a lower aliphatic carboxylic acid ester.

### CROSS-REFERENCE TO RELATED APPLICATION

This application is an application filed under 35 U.S.C. § 111(a) claiming benefit pursuant to 35 U.S.C. § 119(e)(1) of the filing date of the Provisional Application 60/300,866 filed Jun. 27, 2001, pursuant to 35 § 111(b).

### TECHNICAL FIELD

The present invention relates to a method of regenerating a catalyst for use in the production of a lower aliphatic carboxylic acid ester from a lower olefin and a lower aliphatic carboxylic acid.

In particular, the present invention relates to a method of regenerating a catalyst containing a heteropolyacid and/or a heteropolyacid salt, which is used in producing a lower aliphatic carboxylic acid ester by esterifying a lower aliphatic carboxylic acid with a lower olefin, wherein a catalyst, decreased in activity through use, is regenerated.

### BACKGROUND ART

It is well known that a corresponding ester can be produced from a lower aliphatic carboxylic acid and an olefin. Also, it is well known that a catalyst comprising a heteropolyacid and/or a heteropolyacid salt is an effective catalyst for this reaction. Specific examples thereof include those described in Japanese Unexamined Patent Publications No. 4-139148 (JP-A-4-139148), No. 4-139149 (JP-A-4-139149), No. 5-65248 (JP-A-5-65248) and No. 5-294894 (JP-A-5-294894).

Not only the catalyst for use in producing a lower aliphatic carboxylic acid ester from a lower olefin and a lower aliphatic carboxylic acid but also catalysts in general have a “catalyst life” and are usually used within the range of the catalyst life.

As for the “catalyst life”, it is stated, for example, in Shokubai Rekka Mechanism to Rekka Taisaku (Mechanism of Catalyst Deterioration and Measures for Deterioration), 2nd print, page 3, “1.2 Shokubaino Jumyo to wa (What is the Life of Catalyst)”, published by Gijutsu Joho Kyokai K.K. (Jul. 1, 1995) that “The cost of starting material gradually rises accompanying the deterioration of catalyst and the utility cost rises accompanying the purification of the product and the elevation of reaction temperature. Furthermore, the decrease in the mechanical strength of catalyst causes the collapse of catalyst particles and, accompanying the resulting increase in the pressure loss, the utility cost rises. When these costs surpass the sum of the catalyst price and the cost for exchange, the catalyst life is ended. Accordingly, the catalyst life is a function of reaction, reaction conditions, reaction apparatus, plant, economical conditions (catalyst price, cost for exchanging, utility cost) and the like”.

The phenomenon of the “catalyst deterioration” as referred to herein may be variously defined but in a broad sense, means a decrease in the reaction rate. In a narrow sense, the catalyst deterioration includes a decrease in the conversion of starting material, a decrease in the selectivity of objective substance, a decrease in the space time yield and the like.

The catalyst at the end of its life is exchanged for a new catalyst and, for treating the withdrawn catalyst, several methods are known. Specific examples thereof include a method where the withdrawn catalyst is discarded as it is, a method where useful components in the catalyst are recovered and reused, and a method where the catalyst itself is regenerated and reused.

Among these methods, the method where the catalyst is discarded as it is may be simplest, however, depending on the catalyst component, the catalyst cannot be easily discarded or, depending on the kind of catalyst, the discarding cost is high.

The method of recovering useful components has a problem in that the recovery is difficult depending on the kind of catalyst, the treatment of the remaining materials after the recovery raises another problem, and the recovery cost is high.

Therefore, for the treatment of a catalyst at the end of its life, a method of performing an operation called regeneration, to regenerate the catalyst and repeatedly use the catalyst, is most preferred.

The “regeneration of catalyst” means to apply a physical or chemical operation to the deteriorated catalyst to return the catalyst to its initial state or close to its initial state, namely, its initial reaction rate, its initial conversion of starting material, its initial selectivity of objective substance and its initial space time yield. In an industrial process, generally, at least one item selected from the reaction rate, the conversion of starting material, the selectivity of objective substance and the space time yield is used as an index according to the production method and by controlling this item during the process, the production is performed. When the item as an index is decreased, the regeneration of catalyst is generally performed to recover the catalyst.

As for this regeneration method, it is disclosed that the general fixed bed catalyst must be heated in a temperature region higher than the reaction temperature. For example, the regeneration method of the catalyst in various catalyst systems is described in Shokubai Rekka Mechanism to Rekka Taisaku (Mechanism of Catalyst Deterioration and Measures for Deterioration), 2nd print, published by Gijutsu Joho Kyokai K.K. (Jul. 1, 1995)), “2.3 Saisei (Regeneration)” at page 88, “4. (Rekka shokubai no saisei hoho (Regeneration method of deteriorated catalyst)” at pages 184 to 185, “5. Saisei, Kasseika Gijutsu (Regeneration and activation technology)” at page 200, and “5. Shokubai no saisei hoho (Regeneration method of catalyst)” at pages 223 to 225.

In one example among these, regeneration of an Mo-Bi-Fe system catalyst in the ammoxidation of propylene is described, where the catalyst can be regenerated by heating to a temperature of 500 to 800° C. which is higher than the reaction temperature of 420 to 480° C.

However, as for the catalyst comprising a heteropolyacid and/or a heteropolyacid salt for the production of a lower aliphatic carboxylic acid ester from a lower aliphatic carboxylic acid and a lower olefin, which has been rapidly developed in recent years, a regeneration method is not specifically described.

### DISCLOSURE OF INVENTION

The object of the present invention is to provide a method of regenerating a catalyst for use in the production of a lower aliphatic carboxylic acid ester from a lower olefin and a lower aliphatic carboxylic acid, wherein a catalyst decreased in activity accompanying use is effectively regenerated; a catalyst for use in the production of a lower aliphatic carboxylic acid ester, which is regenerated by the regeneration method; and a production process of a lower aliphatic carboxylic acid ester using the catalyst.

The present inventors have made extensive studies on the regeneration method of a catalyst for use in producing a lower aliphatic carboxylic acid ester by reacting a lower olefin and a lower aliphatic carboxylic acid in a gas phase, wherein a catalyst, decreased in activity accompanying use, is effectively regenerated. As a result, it has been found that the catalyst can be effectively regenerated by conducting the regeneration under predetermined conditions. The present invention has been accomplished based on this finding.

More specifically, the present invention (I) provides a method of regenerating a catalyst for use in production of a lower aliphatic carboxylic acid ester by reacting a lower olefin and a lower aliphatic acid ester in a gas phase, which catalyst has been decreased in activity, wherein the catalyst is regenerated by heating.

The present invention (II) provides a process for producing a catalyst for use in production of a lower aliphatic carboxylic acid ester, which comprises, as a step of the process, a step consisting of the method of the present invention (I).

The present invention (III) provides a catalyst for use in production of a lower aliphatic carboxylic acid ester, which has been regenerated by the method of the present invention (I).

The present invention (IV) provides a catalyst for use in production of a lower aliphatic carboxylic acid ester, which has been prepared by the process of the present invention (II).

The present invention (V) provides a process for producing a lower aliphatic carboxylic acid ester comprising reacting a lower olefin and a lower aliphatic carboxylic acid in a gas phase in the presence of the catalyst of the present invention (III) or (IV).

The present invention (VI) provides a process for producing a lower aliphatic carboxylic acid ester by reacting a lower olefin and a lower aliphatic carboxylic acid, which comprises the following steps A and B:

Step A

a step for reacting a lower olefln and a lower aliphatic carboxylic acid in a gas phase in a reactor in which a heteropolyacid and/or heteropolyacid salt supported catalyst is filled to produce a lower aliphatic carboxylic acid ester; and

Step B

a step for conducting the method of the present invention (I) while leaving the heteropolyacid and/or heteropolyacid salt supported catalyst filled in the reactor.

### BEST MODE FOR CARRYING OUT THE INVENTION

The present invention is described in detail below.

The present invention (I) is a method of regenerating a catalyst for use in production of a lower aliphatic carboxylic acid ester by reacting a lower olefin and a lower aliphatic carboxylic acid in a gas phase, wherein the catalyst, which has been decreased in activity through use, is effectively regenerated.

The decrease in activity is caused due to the deterioration of the catalyst. As described in the “Background Art”, the deterioration of the catalyst indicates, in a broad sense, the decrease in the reaction rate and in a narrow sense, includes the decrease in the conversion of starting material, the decrease in the selectivity of objective substance, the decrease in the space time yield and the like.

The temperature where the heating is conducted is preferably from 200 to 450° C., more preferably from 250 to 400° C. If the temperature is less than this range, the regeneration effect may not be easily provided. In known publications, it is stated that generally, the regeneration is preferably conducted at a high temperature, however, in the case of the catalyst used in the present invention (I), if the heating is conducted at a temperature higher than 450° C., the catalyst components may be denatured and the regeneration effect may not be obtained, thus, this is not preferred. In conducting the heating, generation of heat occurs due to the combustion reaction of the carbon content adhering to the catalyst and the temperature inside the catalyst may exceed the set temperature. At this time, since it is not preferred that the temperature of catalyst layer exceeds 450° C., the heating must be conducted, for example, while measuring the temperature inside the catalyst layer.

The atmosphere where the heating is conducted is not particularly limited and any atmosphere may be used. From the standpoint of more effectively combusting and removing the coke adhering to the catalyst, the heating is preferably conducted in an oxidative atmosphere in the presence of water, however, the present invention is not limited thereto.

The method for conducting the heating is not particularly limited and any known method may be used. For example, a method where, while leaving the catalyst filled in the reactor, the reaction is stopped by stopping the introduction of the reaction starting materials such as a lower olefin, a lower aliphatic carboxylic acid and water and after completely desorbing the lower olefin and the lower aliphatic carboxylic acid adsorbed to the catalyst, a gas for the atmosphere for conducting the regeneration is introduced to conduct the heating under the objective conditions, may be used.

Also, a method where the reaction is stopped similarly by stopping the introduction of the reaction starting materials such as a lower olefin, a lower aliphatic carboxylic acid and water and after sufficiently cooling the catalyst layer, the catalyst is withdrawn from the reactor and filled in another vessel to conduct the heating under the objective conditions, may be used. These methods are merely examples and, other than these, any method may be used.

The shape of the reactor or the vessel for heating is not particularly limited and any shape such as vertical or horizontal may be used. Specific examples of the reactor include the “Methanol Shori Kiso Hannoki (Methanol Treating Gaseous Phase Reactor)” shown in FIG. C•4•43 of “4 Kotei Shokubai Kiso Hannoki (Fixed Catalyst Gaseous Phase Reactor)” described at pages 905 to 906 of Kagaku Sochi Binran (Handbook of Chemical Apparatuses), 2nd ed., 3rd print, edited by Kagaku Kogaku Kyokai, published by Maruzen (Feb. 20, 1980)”.

In the case where the catalyst is withdrawn from the reactor, filled in another vessel and then heated, the another vessel is not particularly limited and any vessel may be used insofar as the heating can be performed. Examples thereof include a combustion apparatus and a heating furnace. Examples of the heating furnaces include “heating furnaces of various forms” described in the “Table 4.8.a” at page 453 of Yoshimi Saito, Kagaku Sochi Cost Handbook (Handbook of Chemical Apparatuses Cost), 3rd revised edition, published by Kogyo Chosakai K.K. (Aug. 1, 2000)).

In view of the time and labor necessary for refilling the catalyst or the cost of vessel, as the practical embodiment of the present invention (I), the regeneration is preferably performed while leaving the catalyst filled in the reactor used for producing a lower aliphatic carboxylic acid by reacting a lower olefin and a lower aliphatic carboxylic acid in a gas phase.

The pressure at the time of conducting the heating is not particularly limited and may be an atmospheric pressure or a super-atmospheric pressure. Since the same effect is obtained either under an atmospheric pressure or a super-atmospheric pressure, an atmospheric pressure which is easy to operate is preferred, however, the present invention is not limited thereto.

The time where the heating is conducted is not particularly limited and is preferably from 0.5 to 500 hours, more preferably from 1.0 to 400 hours, most preferably from 1.0 to 300 hours. The optimal time varies depending on the temperature, pressure, catalyst amount and catalyst components at the time of conducting the heating.

Generally, if the heating time is less than 0.5 hours the regeneration effect by heating may not be satisfactorily obtained. If the heating time is prolonged, the effect is liable to increase, however, even if the heating time is prolonged to exceed 500 hours, the effect may not increase any more but the consumption of a utility necessary for the heating may increase and this may be economically not preferred.

The catalyst for use in the present invention is not particularly limited insofar as it is an acid catalyst, however, in particular, a heteropolyacid and/or a heteropolyacid salt are preferred.

The heteropolyacid comprises a center element and a peripheral element to which oxygen is bonded. The center element is usually silicon or phosphorus but may be any atom selected from various atoms belonging to Groups I to XVII of the Periodic Table.

Specific examples of the center element include cupric ion; divalent beryllium, zinc, cobalt and nickel ions; trivalent boron, aluminum, gallium, iron, cerium, arsenic, antimony, phosphorus, bismuth, chromium and rhodium ions; tetravalent silicon, germanium, tin, titanium, zirconium, vanadium, sulfur, tellurium, manganese, nickel, platinum, thorium, hafnium, cerium ions and other rare earth ions; pentavalent phosphorus, arsenic, vanadium and antimony ions; hexavalent tellurium ion; and heptavalent iodide ion, however, the present invention is not limited thereto.

Specific examples of the peripheral element include tungsten, molybdenum, vanadium, niobium and tantalum. However, the present invention is not limited thereto.

These heteropolyacids are also known as a “polyoxoanion”, a “polyoxometallic salt” or a “metal oxide cluster”. Some structures of the well known anions are named after the researcher himself in this field, for example, Keggin, Wells-Dawson or Anderson-Evans-Perloff structure. The heteropolyacid usually has a high molecular weight, for example, a molecular weight of 700 to 8,500, and includes a dimeric complex.

Specific examples of the heteropolyacid which can be used in the catalyst for use in the present invention include:

| | |
|---|---|
| tungstosilicic acid | H₄[SiW₁₂O₄₀].xH₂O |
| tungstophosphoric acid | H₃[PW₁₂O₄₀].xH₂O |
| molybdophosphoric acid | H₃[PMo₁₂O₄₀].xH₂O |
| molybdosilicic acid | H₄[SiMo₁₂O₄₀].xH₂O |
| vanadotungstosilicic acid | H₄₊ₙ[SiVW₁₂₋ₙO₄₀].xH₂O |
| vanadotungstophosphoric acid | H₃₊ₙ[PVₙW₁₂₋ₙO₄₀].xH₂O |
| vanadomolybdophosphoric acid | H₃₊ₙ[PVₙMo₁₂₋ₙO₄₀].xH₂O |
| vanadomolybdosilicic acid | H₄₊ₙ[SiVₙMo₁₂₋ₙO₄₀].xH₂O |
| molybdotungstosilicic acid | H₄[SiMoₙW₁₂₋ₙO₄₀].xH₂O |
| molybdotungstophosphoric acid | H₃[PMoₙW₁₂₋ₙO₄₀].xH₂O |

wherein n is an integer of 1 to 11 and x is an integer of 1 or more. Of course, the present invention is not limited thereto.

Among these, preferred are tungstosilicic acid, tungstophosphoric acid, molybdophosphoric acid, molybdosilicic acid, vanadotungstosilicic acid, vanadotungstophosphoric acid and molybdotungstophosphoric acid, more preferred are tungstosilicic acid, tungstophosphoric acid, vanadotungstosilicic acid and vanadotungstophosphoric acid.

The method for synthesizing these heteropolyacids is not particularly limited and any method may be used. For example, the heteropolyacid can be obtained by heating an acidic aqueous solution (pH: approximately from 1 to 2) containing a salt of molybdic acid or tungstic acid and a simple oxygen acid of a hetero atom or a heteropolyacid salt. For isolating the heteropolyacid compound from the resulting aqueous heteropolyacid solution, a method of crystallizing and separating the compound as a metal salt may be used. Specific examples thereof are described in Shin Jikken Kagaku Koza 8, Muki Kagobutsuno Gosei (III) (New Experimental Chemistry Course 8, Synthesis (III) of Inorganic Compounds), 3rd ed., edited by Nippon Kagaku Kai, published by Maruzen, page 1413 (Aug. 20, 1984), however, the present invention is not limited thereto.

The Keggin structure of the heteropolyacid synthesized can be identified by the X-ray diffraction or UV or IR measurement in addition to the chemical analysis.

The heteropolyacid salt is not particularly limited insofar as it is a metal salt or onium salt resulting from displacing a part or all of the hydrogen atoms of the heteropolyacid. Specific examples thereof include metal salts such as of lithium, sodium, magnesium, barium, copper, gold and gallium and onium salts, however, the present invention is not limited thereto.

Among these, preferred are lithium salts, sodium salts, gallium salts, copper salts and gold salts, more preferred are lithium salts, sodium salts and copper salts.

Examples of the starting material for the element of forming the heteropolyacid salt in the present invention include lithium nitrate, lithium acetate, lithium sulfate, lithium sulfite, lithium carbonate, lithium phosphate, lithium oxalate, lithium nitrite, lithium chloride, lithium citrate, sodium nitrate, sodium acetate, sodium sulfate, sodium carbonate, monosodium phosphate, disodium phosphate, sodium oxalate, sodium nitrite, sodium chloride, sodium citrate, magnesium nitrate hexahydrate, magnesium acetate tetrahydrate, magnesium sulfate, magnesium carbonate, magnesium phosphate tricosahydrate, magnesium oxalate dihydrate, magnesium chloride, magnesium citrate, barium nitrate, barium acetate, barium sulfate, barium carbonate, barium hydrogenphosphate, barium oxalate monohydrate, barium sulfite, barium chloride, barium citrate, copper nitrate, copper acetate, copper sulfate, copper carbonate, copper diphosphate, copper oxalate, copper chloride, copper citrate, aurous chloride, chloroauric acid, auric oxide, auric hydroxide, auric sulfide, aurous sulfide, gallium dichloride, gallium monochloride, gallium citrate, gallium acetate, gallium nitrate, gallium sulfate, gallium phosphate, ammonium acetate, ammonium carbonate, ammonium nitrate, ammonium dihydrogenphosphate, ammonium hydrogencarbonate, ammonium citrate, ammonium nitrate, diammonium phosphate, monoammonium phosphate and ammonium sulfate, however, the present invention is not limited thereto.

Among these, preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate, copper citrate, aurous chloride, chloroauric acid, gallium citrate, gallium acetate and gallium nitrate, and more preferred are lithium nitrate, lithium acetate, lithium carbonate, lithium oxalate, lithium citrate, sodium nitrate, sodium acetate, sodium carbonate, sodium oxalate, sodium citrate, copper nitrate, copper acetate, copper carbonate and copper citrate.

Specific examples of the heteropolyacid salt for use in the present invention include lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid, gallium salt of vanadotungstophosphoric acid, lithium salt of vanadomolybdophosphoric acid, sodium salt of vanadomolybdophosphoric acid, copper salt of vanadomolybdophosphoric acid, gold salt of vanadomolybdophosphoric acid, gallium salt of vanadomolybdophosphoric acid, lithium salt of vanadomolybdosilicic acid, sodium salt of vanadomolybdosilicic acid, copper salt of vanadomolybdosilicic acid, gold salt of vanadomolybdosilicic acid, gallium salt of vanadomolybdosilicic acid, lithium salt of molybdotungstophosphoric acid, sodium salt of molybdotungstophosphoric acid, copper salt of molybdotungstophosphoric acid, gold salt of molybdotungstophosphoric acid and gallium salt of molybdotungstophosphoric acid.

Among these, preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of molybdophosphoric acid, sodium salt of molybdophosphoric acid, copper salt of molybdophosphoric acid, gold salt of molybdophosphoric acid, gallium salt of molybdophosphoric acid, lithium salt of molybdosilicic acid, sodium salt of molybdosilicic acid, copper salt of molybdosilicic acid, gold salt of molybdosilicic acid, gallium salt of molybdosilicic acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid, lithium salt of molybdotungstophosphoric acid, sodium salt of molybdotungstophosphoric acid, copper salt of molybdotungstophosphoric acid, gold salt of molybdotungstophosphoric acid and gallium salt of molybdotungstophosphoric acid.

More preferred are lithium salt of tungstosilicic acid, sodium salt of tungstosilicic acid, copper salt of tungstosilicic acid, gold salt of tungstosilicic acid, gallium salt of tungstosilicic acid, lithium salt of tungstophosphoric acid, sodium salt of tungstophosphoric acid, copper salt of tungstophosphoric acid, gold salt of tungstophosphoric acid, gallium salt of tungstophosphoric acid, lithium salt of vanadotungstosilicic acid, sodium salt of vanadotungstosilicic acid, copper salt of vanadotungstosilicic acid, gold salt of vanadotungstosilicic acid, gallium salt of vanadotungstosilicic acid, lithium salt of vanadotungstophosphoric acid, sodium salt of vanadotungstophosphoric acid, copper salt of vanadotungstophosphoric acid, gold salt of vanadotungstophosphoric acid and gallium salt of vanadotungstophosphoric acid.

The heteropolyacid has a relatively high solubility particularly in a polar solvent such as water or an oxygen-containing solvent and the solubility thereof can be controlled by selecting a suitable counter ion.

In the catalyst for use in the present invention (I), the heteropolyacid and/or heteropolyacid salt as a catalytic activity component may or may not be supported on a support and this is not particularly limited. In the case where the heteropolyacid and/or heteropolyacid salt is supported, a support is used. The substance which can be used as the support is not particularly limited and a porous substance commonly used as a support may be used. Specific examples thereof include those comprising silica gel, diatomaceous earth, montmorillonite, titania, activated carbon, alumina or silica alumina, preferably silica gel or titania.

The shape of the support is not limited and the support may be in the powder, spherical, pellet-like or any other form. A support in the spherical or pellet-like form is preferred.

Furthermore, the particle size is not particularly limited and although the preferred particle size varies depending on the reaction form, the average diameter is preferably from 2 to 10 mm in the case of a fixed bed reaction and preferably from powder to 5 mm in the case of a fluid bed reaction.

The support is most preferably a spherical or pellet-like silica gel support, however, the present invention is not limited thereto.

The production process of the catalyst for use in the production of a lower aliphatic carboxylic acid ester, which is used in the present invention (I), is not particularly limited and a known method may be used. Specifically, the catalyst is obtained by loading a solution or suspension of heteropolyacid and/or heteropolyacid salt on a support. For example, a heteropolyacid is dissolved in distilled water corresponding to the liquid absorption amount of a support used and the solution is impregnated into the support, whereby the catalyst can be prepared. The catalyst may also be prepared by using an aqueous solution of excess heteropolyacid, impregnating it into a support while appropriately moving the support in the heteropolyacid solution and then removing excess acid by filtration. At this time, the volume of the solution or suspension varies depending on the support used or the loading method.

The solvent used here is not particularly limited insofar as it can uniformly dissolve or suspend the desired heteropolyacid and/or heteropolyacid salt, and examples of the solvent which can be used include water, an organic solvent and a mixture thereof, however, the present invention is not limited thereto.

Also, the dissolving or suspending method may not be particularly limited as far as the desired heteropolyacid and/or heteropolyacid salt can be uniformly dissolved or suspended. In the case of a free acid, a free acid which can dissolve may be dissolved as it is in a solvent and even in the case of a free acid which cannot completely dissolve, if the free acid can be uniformly suspended by forming it into fine powder, the free acid may be suspended as such.

In the case of a heteropolyacid salt, a heteropolyacid and a starting material salt of a neutralizing element are dissolved together or separately and then mixed, whereby a uniform solution or suspension can be obtained.

The thus-obtained wet catalyst is suitably dried by placing it in a heating oven for a few hours. The drying method is not particularly limited and any method such as stationary system or belt conveyor system may be used. After the drying, the catalyst is preferably cooled to the ambient temperature in a desiccator so as not to absorb moisture.

The amount of the heteropolyacid and/or heteropolyacid salt supported in the heteropolyacid supported catalyst prepared can be simply calculated by subtracting the weight of the support used from the weight after drying of the catalyst prepared. The amount supported may be more exactly measured by a chemical analysis such as inductively coupled plasma emission spectrometry (hereinafter referred to as “ICP”), fluorescent X-ray method or atomic absorption method.

The amount of the heteropolyacid and/or heteropolyacid salt supported is preferably from 10 to 150 mass %, more preferably from 30 to 100 mass %, based on the weight of the support.

If the heteropolyacid and/or heteropolyacid salt content is less than 10 mass %, the content of active components in the catalyst is too small so that the activity per catalyst unit weight may disadvantageously decrease, whereas if the heteropolyacid and/or heteropolyacid salt content exceeds 200 mass %, the effective pore volume decreases so that the effect by the increase in the supported amount may not be provided and at the same time, coking may disadvantageously occur to greatly shorten the catalyst life.

The mechanism of regeneration effect by the heating has not been elucidated in detail, however, it is confirmed that when the bulk density of the catalyst is measured before reaction, after decrease in the activity and after regeneration, the catalyst after decrease in the activity increases in the bulk density more than the catalyst before reaction and the bulk density decreases after regeneration. Accordingly, it is considered that the activity is decreased due to coke generated by the reaction and attached to the catalyst and the coke is removed by the heating for regeneration. However, if the heating is conducted at a temperature higher than 450° C., the bulk density may be decreased similarly to the case of conducting the heating at 450° C. or less but the activity may not be recovered. From this, it is presumed that the catalyst component may be denatured at a temperature higher than 450° C.

The present invention (II) is a process for producing a catalyst for use in production of a lower aliphatic carboxylic acid ester, which comprises, as a step of the process, a step consisting of the method of the present invention (I).

The present invention (III) is a catalyst for use in production of a lower aliphatic carboxylic acid ester, which has been regenerated by the method of the present invention (I).

The present invention (IV) is a catalyst for use in production of a lower aliphatic carboxylic acid ester, which has been prepared by the process of the present invention (II).

In the method of regenerating the catalyst performed in the present invention (III) or (IV) where a catalyst for the production of a lower aliphatic carboxylic acid ester is heated, the heating temperature, the heating atmosphere, the heating operation method, the vessel used at the heating, the pressure at the heating and the heating time are the same as those in the present invention (I).

Also, the heteropolyacid and/or heteropolyacid salt for use in the catalyst for the production of a lower aliphatic carboxylic acid ester, the support, the method of loading the heteropolyacid and/or heteropolyacid salt on the support, the method for measuring the amount of heteropolyacid and/or heteropolyacid salt supported and the preferred amount of heteropolyacid and/or heteropolyacid salt supported, which are used in the present invention (III) or (IV), are the same as those in the present invention (I).

The present invention (V) is a process for producing a lower aliphatic carboxylic acid ester comprising reacting a lower olefin and a lower aliphatic carboxylic acid in a gas phase in the presence of the catalyst of the present invention (III) or (IV).

In the method of regenerating the catalyst performed in the present invention (V) where a catalyst for the production of a lower aliphatic carboxylic acid ester is heated, the heating temperature, the heating atmosphere, the heating operation method, the vessel used at the heating, the pressure at the heating and the heating time are the same as those in the present invention (I).

The heteropolyacid and/or heteropolyacid salt for use in the catalyst for the production of a lower fatty carboxylic acid ester, the support, the method of loading the heteropolyacid and/or heteropolyacid salt on the support, the method for measuring the amount of heteropolyacid and/or heteropolyacid salt supported and the preferred amount of heteropolyacid and/or heteropolyacid salt supported, which are used in the present invention (III), are the same as those in the present invention (I).

In practicing the production process of a lower aliphatic carboxylic acid ester of the present invention (V), the reaction form of the gas phase reaction is not particularly limited. The reaction may be performed in any form such as fixed bed or fluidized bed. Also, the shape of the support, which governs the shape or size of the catalyst, may be selected from those formed into a size from powder to a few mm in size according to the reaction form employed.

In the production process of a lower fatty carboxylic acid ester of the present invention (V), examples of the lower olefin which can be used include ethylene, propylene, n-butene, isobutene and a mixture of two or more thereof. Among these, preferred are ethylene and propylene, more preferred is ethylene.

Examples of the lower aliphatic carboxylic acid which can be used include formic acid, acetic acid, propionic acid, butyric acid, acrylic acid, methacrylic acid and mixture of two or more thereof. Among these, preferred are acetic acid, butyric acid, propionic acid and acrylic acid, more preferred is acetic acid.

In the production process of a lower aliphatic carboxylic acid ester of the present invention (V), specific preferred examples of the combination of a lower olefin and a lower aliphatic carboxylic acid include ethylene and acetic acid, ethylene and propionic acid, propylene and acetic acid, and propylene and propionic acid. Most preferred is the combination of ethylene and acetic acid.

The ratio between the lower olefin and the lower aliphatic carboxylic acid used as starting materials is not particularly limited. From the standpoint of conversion of the lower olefin, the lower olefin is preferably used in an amount equimolar to, or in excess of, the amount of the lower aliphatic carboxylic acid. Specifically, the ratio of lower olefin:lower aliphatic carboxylic acid is preferably, in terms of a molar ratio, from 1:1 to 30:1, more preferably 3:1 to 20:1, still more preferably from 5:1 to 15:1.

The addition of water to starting materials is not particularly limited and water may or may not be added. The addition of water in a small amount is preferred from the standpoint of maintaining the catalyst activity. However, if an excessively large amount of water is added, the amount of by-products produced, such as alcohol and ether, disadvantageously increases.

The preferred reaction conditions such as temperature and pressure in the production process of a lower aliphatic carboxylic acid ester of the present invention (V) vary depending on the lower olefin and lower aliphatic carboxylic acid used as the starting materials. The reaction conditions such as temperature and pressure are preferably combined so that the starting materials can be kept gaseous and the reaction can satisfactorily proceed.

Generally, the temperature is preferably from 120 to 300° C., more preferably from 140 to 250° C. The pressure is preferably from atmospheric pressure to 3 MPaG (gauge pressure), more preferably from atmospheric pressure to 2 MPaG (gauge pressure).

The gas hourly space velocity (hereinafter simply referred to as “GHSV”) of starting materials is not particularly limited, however, if the space velocity is too large, the starting materials pass through before the reaction sufficiently proceeds, whereas if it is too small, there may arise problems, for example, the productivity may decrease. The GHSV is preferably from 100 to 7,000 hr⁻¹, more preferably from 300 to 3,000 hr⁻¹.

The unreacted lower olefin and by-products in the reaction, such as alcohol and ether, each can be recycled as they are.

The present Invention (VI) is a process for producing a lower aliphatic carboxylic acid ester by reacting a lower olefin and a lower aliphatic carboxylic acid, which comprises the following steps A and B:

Step A

a step for reacting a lower olefln and a lower aliphatic carboxylic acid in a gas phase in a reactor in which a heteropolyacid and/or heteropolyacid salt supported catalyst is filled to produce a lower aliphatic carboxylic acid ester; and

Step B

a step for conducting the method of the present invention (I) while leaving the heteropolyacid and/or heteropolyacid salt supported catalyst filled in the reactor.

The step A is described below. The step A is a step for reacting a lower olefin and a lower aliphatic carboxylic acid using a heteropolyacid and/or heteropolyacid salt supported catalyst to produce a lower aliphatic carboxylic acid ester. The heteropolyacid and/or heteropolyacid salt supported catalyst used in this step can be obtained by the method hereinbefore described with respect to the present invention (I). The heteropolyacid and/or heteropolyacid salt supported catalyst used here is not particularly limited and may or may not be regenerated by the method of the present invention (I). The reaction form practiced in the step A, the lower olefin which can be used, the lower fatty carboxylic acid which can be used, the combination of lower olefin and lower fatty carboxylic acid which can be used, the ratio of starting materials used, the addition of water, the temperature, the pressure, GHSV of starting materials and the treatment of unreacted materials are the same as those in the present invention (V).

The step B is described below. The step B is a step for conducting the regeneration by heating the heteropolyacid and/or heteropolyacid salt supported catalyst while leaving the catalyst filled in the reactor. The method of conducting this step is not particularly limited and any method may be used. For example, a method where while leaving the catalyst filled in the reactor, the reaction is stopped by stopping the introduction of the reaction starting materials such as a lower olefin, a lower aliphatic carboxylic acid and water and after completely desorbing the lower olefin and the lower aliphatic carboxylic acid adsorbed to the catalyst, a gas for the atmosphere for conducting the regeneration is introduced to conduct the regeneration under the objective regeneration conditions, may be used.

By disposing a plurality of reactors in parallel, the step A, the step B and other steps may be performed at the same time in respective reactors. Or, the step A, the step B and other steps may be separately performed in individual reactors.

The order of performing the step A and the step B is not particularly limited and either step may be first performed. Also, the step A and the step B may be repeatedly performed.

The present invention is further illustrated below by referring to the Examples and Comparative Examples, however, these Examples are only for illustrating the present invention and the present invention should not be construed as being limited thereto.

Supports each having the following properties were used.

Support 1:

synthetic silica (N-602A, produced by Nikki Kagaku K.K.) (specific surface area: 298 m²/g, pore volume: 0.91 cm³/g)

Support 2:

synthetic silica (CARiACT Q-10, produced by Fuji Silysia Chemical Ltd.) (specific surface area: 334.6 m²/g, pore volume: 0.99 cm³/g)

Support 3:

natural silica (KA-0, produced by Süd-chemie AG) (specific surface area: 98.2 m²/g, pore volume: 0.28 cm³/g)

Support 4:

natural silica (KA-1, produced by Süd-chemie AG) (specific surface area: 111.9 m²/g, pore volume: 0.42 cm³/g)

Support 5:

titania (Cs-300S-46, produced by Sakai Kagaku Kogyo K.K.) (specific surface area: 77 m²/g, pore volume: 0.39 cm³/g)

Preparation Method of Catalyst 1

The support 1 was preliminarily dried for 4 hours by a (hot air) dryer previously adjusted to 110° C. 1 L of the preliminarily dried support 1 was measured on the bulk density by a 1-L measuring cylinder. Tungstosilicic acid and lithium nitrate were weighed each to the amount shown in Table 1, 15 ml of pure water was added thereto, and the mixture was uniformly dissolved. This impregnating solution was diluted with pure water to the Volume of Prepared Solution shown in Table 1. Thereafter, the preliminarily dried support was weighed to the amount shown in Table 1, added to the impregnating solution and thoroughly stirred, thereby impregnating the solution into the support. The support impregnated with the solution was transferred to a porcelain dish, air dried for 1 hour and then dried for 5 hours by a hot air dryer adjusted to 150° C. After the drying, the support was transferred into a desiccator and allowed to cool to room temperature. The weight of the catalyst obtained was measured.

Preparation Method of Catalysts 2 to 5

The preparation was carried out by changing the kind and weight of the support, the kind and weight of each catalyst component and the kind and weight of the neutralization salt as shown in Table 1 in Preparation Method of Catalyst 1.

TABLE 1

| | | | Weight | | Amount of | Kind of | | Volume of | Weight | |
|---|---|---|---|---|---|---|---|---|---|---|
| | | Bulk | of | Kind of | Catalyst | Neutral- | Amount of | Prepared | After | |
| Name of | | Density | Support | Catalyst | Component | ization | Neutralization | Solution | Drying | Name of |
| Catalyst | Support | (g/L) | (g) | Component | (g) | Salt | Salt (g) | (ml) | (g) | Support |
| Catalyst 1 | Support 1 | 448 | 44.8 | HSiW | 40.70 | LiNO₃ | 0.0838 | 41 | 80.8 | N-602A |
| Catalyst 2 | Support 2 | 456 | 45.6 | HSiW | 34.88 | none | none | 43 | 75.8 | Q-10 |
| Catalyst 3 | Support 3 | 558 | 55.8 | HPW | 53.44 | none | none | 34 | 104.2 | KA-0 |
| Catalyst 4 | Support 4 | 573 | 57.3 | HPWE | 65.33 | LiNO₃ | 0.1317 | 33 | 114.5 | KA-1 |
| Catalyst 5 | Support 5 | 822 | 82.2 | HSiW | 40.70 | NaOAc | 0.0988 | 31 | 116.7 | Cs-300S-46 |

HPW: H₃PW₁₂O₄₀
HSiW: H₄SiW₁₂O₄₀

### EXAMPLE 1

Into an SUS316L reactor, 100 ml of the catalyst obtained in the Preparation Method of Catalyst 1 was filled. Thereinto, a mixed gas at a volume ratio of ethylene:acetic acid:water vapor:nitrogen=78.5 8.0:4.5:9.0 was introduced at a temperature of 165° C., a pressure of 0.8 MPaG (gauge pressure) and a GHSV of 1,500 hr⁻¹ and reacted. The gas passed through the catalyst layer was collected under ice cooling for a predetermined time and the entire amount of the obtained solution (hereinafter called a “condensed solution”) was recovered and analyzed. The outlet gas remaining uncondensed (hereinafter called an “uncondensed gas”) was measured on the gas flow rate for the same predetermined time as in the condensed solution and then 50 ml of the gas was sampled and analyzed. The space time yield (hereinafter simply referred to as “STY”) after the passage of 5 hours from the starting of reaction is called an initial activity. The STY after the activity was decreased by the reaction is shown in the column of “STY of Ethyl Acetate” of Table 2.

40 ml of the catalyst after the decrease of activity was left in the reactor and the residue was withdrawn from the reactor. The catalyst withdrawn was subjected to the measurement of the bulk density. While leaving 40 ml of catalyst filled in the reactor, the regeneration was performed under the conditions shown in the column of “Regeneration Conditions” of Table 2. After the regeneration, the reaction and the analysis were carried out in the same manner as above. The results are shown in Table 3. The STY in the initial stage of reaction was 226 (g/(L·hr)), the STY after the decrease of activity was 29 (g/(L·hr)), and the STY after the regeneration was 158 (g/(L·hr)).

TABLE 2

| | | | | Regeneration Conditions | | | | |
|---|---|---|---|---|---|---|---|---|
| | | STY of Ethyl Acetate | | Composition of Gas | | | | |
| | | Initial | After | Treated, | Gas Flow | | Treating | Treating |
| | | Activity (g · | Decrease of Activity | Oxygen:Water:Nitrogen | Rate | Pressure | Temperature | Time |
| | Catalyst | L⁻¹ · hr⁻¹) | (g · L⁻¹ · hr⁻¹) | (by mol) | (NL/hr) | (MPaG) | (C°) | (hr) |
| Example 1 | Catalyst 1 | 226 | 29 | 7:4:89 | 60 | 0.8 | 250 | 240 |
| Example 2 | Catalyst 1 | 226 | 29 | 79:0:21 | | 0 | 350 | 24 |
| Example 3 | Catalyst 2 | 265 | 30 | 79:0:21 | | 0 | 300 | 96 |
| Example 4 | Catalyst 2 | 265 | 30 | 79:0:21 | | 0 | 350 | 6 |
| Example 5 | Catalyst 3 | 191 | 59 | 7:4:89 | 40 | 0.6 | 210 | 48 |
| Example 6 | Catalyst 3 | 191 | 59 | 79:0:21 | | 0 | 250 | 96 |
| Example 7 | Catalyst 3 | 191 | 59 | 79:0:21 | | 0 | 250 | 240 |
| Example 8 | Catalyst 3 | 191 | 59 | 79:0:21 | | 0 | 350 | 6 |
| Example 9 | Catalyst 3 | 191 | 59 | 79:0:21 | | 0 | 400 | 6 |
| Example 10 | Catalyst 4 | 200 | 55 | 0:10:90 | 40 | 0.2 | 250 | 24 |
| Example 11 | Catalyst 5 | 176 | 39 | 0:0:100 | 60 | 0.4 | 250 | 24 |
| Comparative | Catalyst 1 | 226 | 29 | 7:4:89 | 60 | 0.4 | 150 | 48 |
| Example 1 | | | | | | | | |
| Comparative | Catalyst 2 | 265 | 30 | 79:0:21 | | 0 | 500 | 6 |
| Example 2 | | | | | | | | |
| Comparative | Catalyst 3 | 191 | 59 | 0:0:100 | 60 | 0.8 | 170 | 12 |
| Example 3 | | | | | | | | |
| Comparative | Catalyst 4 | 200 | 55 | 0:10:90 | 40 | 0.2 | 170 | 24 |
| Example 4 | | | | | | | | |
| Comparative | Catalyst 5 | 200 | 55 | 79:0:21 | | 0 | 500 | 6 |
| Example 5 | | | | | | | | |

TABLE 3

| | | Bulk Density (g/L) | | | Reaction Results | |
|---|---|---|---|---|---|---|
| | | | After | | | STY of Ethyl |
| | | Before | Decrease of | After | Reaction | Acetate |
| | Catalyst | Reaction | Activity | Regeneration | Time (hr) | (g · L⁻¹ · hr⁻¹) |
| Example 1 | Catalyst 1 | 808 | 858 | Not measurable | 5 | 158 |
| Example 2 | Catalyst 1 | 880 | 858 | 762 | 5 | 167 |
| Example 3 | Catalyst 2 | 758 | 803 | 731 | 5 | 211 |
| Example 4 | Catalyst 2 | 758 | 803 | 752 | 5 | 187 |
| Example 5 | Catalyst 3 | 1042 | 1077 | Not measurable | 5 | 126 |
| Example 6 | Catalyst 3 | 1042 | 1077 | 1034 | 5 | 181 |
| Example 7 | Catalyst 3 | 1042 | 1077 | 1031 | 5 | 189 |
| Example 8 | Catalyst 3 | 1042 | 1077 | 1002 | 5 | 193 |
| Example 9 | Catalyst 3 | 1042 | 1077 | 996 | 5 | 171 |
| Example 10 | Catalyst 4 | 1145 | 1228 | Not measurable | 5 | 100 |
| Example 11 | Catalyst 5 | 1167 | 1198 | Not measurable | 5 | 98 |
| Comparative | Catalyst 1 | 808 | 858 | Not measurable | 5 | 42 |
| Example 1 | | | | | | |
| Comparative | Catalyst 2 | 758 | 803 | 797 | 5 | 5 |
| Example 2 | | | | | | |
| Comparative | Catalyst 3 | 1042 | 1077 | Not measurable | 5 | 58 |
| Example 3 | | | | | | |
| Comparative | Catalyst 4 | 1145 | 1228 | Not measurable | 5 | 59 |
| Example 4 | | | | | | |
| Comparative | Catalyst 5 | 1167 | 1198 | 1157 | 5 | 3 |
| Example 5 | | | | | | |

### EXAMPLE 2

The catalyst withdrawn after the reaction in Example 1 was placed in a muffle furnace and regenerated under the conditions shown in the column of “Regeneration Conditions” of Table 2. After the treatment, the catalyst was cooled to room temperature and measured on the bulk density. 40 ml of the regenerated catalyst was filled in a reaction tube, and the reaction and the analysis were carried out under the same conditions as in Example 1. The results are shown in Table 2.

### EXAMPLES 3 AND 4

The reaction and the analysis were performed under the same conditions as in Example 1 except for changing the catalyst from Catalyst 1 to Catalyst 2. After the completion of reaction, the entire amount of the catalyst was withdrawn from the reactor. The catalyst withdrawn was measured on the bulk density. Every 50 ml of the catalyst was placed in a muffle furnace and regenerated under the conditions shown in “Regeneration Conditions” of Table 2. After the treatment, the catalyst was cooled to room temperature and measured on the bulk density. 40 ml of the regenerated catalyst was filled in a reaction tube, and the reaction and the analysis were carried out under the same conditions as in Example 1. The results are shown in Table 3.

### EXAMPLE 5

The reaction and the analysis were performed under the same conditions as in Example 1 except for changing the catalyst from Catalyst 1 to Catalyst 3 and changing the catalyst amount from 100 ml to 250 ml. 40 ml of the catalyst after the decrease of activity was left in the reactor and the residue was withdrawn from the reactor. The catalyst withdrawn was measured on the bulk density. While leaving 40 ml of catalyst filled in the reactor, the regeneration was performed under the conditions shown in the column of “Regeneration Conditions” of Table 2. After the regeneration, the reaction and the analysis were carried out in the same manner as above. The results are shown in Table 3.

### EXAMPLES 6 TO 9

Every 50 ml of the catalyst withdrawn after the reaction in Example 5 was placed in a muffle furnace and regenerated under the conditions shown in the column of “Regeneration Conditions” of Table 2. After the treatment, the catalyst was cooled to room temperature and measured on the bulk density. 40 ml of the regenerated catalyst was filled in a reaction tube, and the reaction and the analysis were carried out under the same conditions as in Example 1. The results are shown in Table 3.

### EXAMPLES 10 AND 11

The reaction and the analysis were performed under the same conditions as in Example 1 except for changing the catalyst from Catalyst 1 to Catalyst 4 or Catalyst 5 and changing the catalyst amount from 100 ml to 40 ml. While leaving the catalyst after the decrease of activity filled in the reactor, the regeneration was carried out by the method shown in the column of “Regeneration Conditions” of Table 2. Thereafter, the reaction and the analysis were again carried out. The results are shown in Table 3.

### COMPARATIVE EXAMPLE 1

40 ml of the same catalyst as in Example 1 was filled in a reaction tube and thereinto, a mixed gas at a volume ratio of ethylene:acetic acid:water vapor nitrogen=78.5:8.0:4.5:9.0 was introduced at a temperature of 165° C., a pressure of 0.8 MPaG (gauge pressure) and a GHSV of 1,500 hr⁻¹ and reacted. The gas passed through the catalyst layer was collected and analyzed in the same manner as in Example 1. The results are shown in Table 2.

While leaving the catalyst filled in the reactor, the regeneration was carried out under the conditions shown in the column of “Regeneration Conditions” of Table 2. After the regeneration, the reaction and the analysis were again carried out under the above-described conditions. The results are shown in Table 3.

### COMPARATIVE EXAMPLES 2 AND 5

The reaction was performed in the same manner as in Comparative Example 1 except for changing Catalyst 1 to Catalyst 2 or Catalyst 5. The catalyst after the reaction was withdrawn from the reactor, the catalyst withdrawn was placed in a muffle furnace and regenerated under the conditions shown in the column of “Regeneration Conditions” of Table 2. After the regeneration, the catalyst was cooled to room temperature and measured on the bulk density. 40 ml of the regenerated catalyst was filled in a reaction tube, and the reaction and the analysis were carried out under the same conditions as in Example 1. The results are shown in Table 3.

### COMPARATIVE EXAMPLES 3 AND 4

The reaction, the regeneration and the analysis were carried out in the same manner as in Comparative Example 1 except for changing Catalyst 1 to Catalyst 3 or Catalyst 4. The results are shown in Table 3.

The analysis was carried out under the following conditions.

Conditions for Analysis of Uncondensed Gas

An absolute calibration curve method was used for the analysis. The analysis was carried out under the following conditions by sampling 50 ml of the effluent gas and allowing the whole amount to flow into a 1 ml-volume gas sampler attached to a gas chromatograph.

1. Ether, Carboxylic Acid Ester, Alcohol, Trace By-Products

Gas chromatograph:

gas chromatograph (GC-14B, manufactured by Shimadzu Corporation) with a gas sampler (MGS-4, measuring tube: 1 ml) for Shimadzu gas chromatograph

Column:

packed column SPAN80 15% Shinchrom A, 60 to 80 mesh (length: 5 m)

Carrier gas:

nitrogen (flow rate: 25 ml/min)

Temperature conditions:

the detector and the vaporization chamber were at 120° C. and the column was at a constant temperature of 65° C.

Detector:

FID (H₂ pressure: 60 kPa, air pressure: 100 kPa)

2. Hydrocarbon

Gas chromatograph:

gas chromatograph (GC-14B, manufactured by Shimadzu Corporation) with a gas sampler (MGS-4, measuring tube: 1 ml) for Shimadzu gas chromatograph

Column:

packed column Unicarbon A-400, 80/100 mesh, length: 2 m

Carrier gas:

helium (flow rate: 23 ml/min)

Temperature conditions:

the detector and the vaporization chamber were at 130° C. and the column temperature was elevated from 40° C. to 95° C. at a temperature rising rate of 40° C./min

Detector:

FID (H₂ pressure: 70 kPa, air pressure: 100 kPa)

3. Ethylene

Gas chromatograph:

gas chromatograph (GC-14B, manufactured by Shimadzu Corporation) with a gas sampler (MGS-4, measuring tube: 1 ml) for Shimadzu gas chromatograph

Column:

packed column Unibeads IS, length: 3 m

Carrier gas:

helium (flow rate: 20 ml/min)

Temperature conditions:

the detector and the vaporization chamber were at 120° C. and the column was at a constant temperature of 65° C.

Detector:

TCD (He pressure: 70 kPa, current: 90 mA, temperature: 120° C.)

Analysis of Collected Solution

The internal standard method was used for the analysis. An analysis solution was prepared by adding 1 ml of 1,4-dioxane as the internal standard substance to 10 ml of the reaction solution collected and 0.4 μl of the analysis solution was injected into a gas chromatograph.

Gas chromatograph:

GC-14B, manufactured by Shimadzu Corporation

Column:

capillary column TC-WAX (length: 30 m, internal diameter: 0.25 mm, film thickness: 0.25 μm)

Carrier gas:

nitrogen (split ratio: 20, column flow rate: 1 ml/min)

Temperature conditions:

the detector and the vaporization chamber were at 200° C. and the column was kept at 40° C. for 7 minutes from the initiation of analysis, thereafter elevated up to 230° C. at a temperature rising rate of 10° C./min, and kept at 230° C. for 5 minutes

Detector:

FID (H₂ pressure: 70 kPa, air pressure: 100 kPa)

### INDUSTRIAL APPLICABILITY

According to the present invention, a catalyst for use in the production of a lower aliphatic carboxylic acid ester from a lower olefin and a lower aliphatic carboxylic acid, which catalyst has been reduced in activity through use, is heated so that the activity can be recovered and the catalyst can be reused.

1. A method of regenerating a catalyst for use in production of a lower aliphatic carboxylic acid ester by reacting a lower olefin and a lower aliphatic acid ester in a gas phase, which catalyst has been decreased in activity, wherein the catalyst is regenerated by heating.

2. A method according to claim 1, wherein the heating is conducted at a temperature of 200 to 450° C.

3. A method according to claim 1 or 2, wherein the heating is conducted in an oxidative atmosphere.

4. A method according to any one of claims 1 to 3, wherein the heating is conducted in the presence of water.

5. A method according to any one of claims 1 to 4, wherein the heating is conducted in a reactor used for the reaction of a lower olefin and a lower aliphatic acid ester to produce a lower aliphatic carboxylic acid ester.

6. A method according to any one of claims 1 to 5, wherein the catalyst is a supported catalyst.

7. A method according to any one of claims 1 to 6, wherein the catalyst contains a heteropolyacid and/or a heteropolyacid salt.

8. A method according to claim 7, wherein the heteropolyacid is selected from the group consisting of tungstosilicic acid, tungstophosphoric acid, molybdophosphoric acid, molybdosilicic acid, vanadotungstosilicic acid, vanadotungstophosphoric acid, vanadomolybdophosphoric acid, vanadomolybdosilicic acid, molybdotungstosilicic acid and molybdotungstophosphoric acid.

9. A method according to claim 7, wherein the heteropolyacid salt is selected from the group consisting of lithium salts, sodium salts, magnesium salts, barium salts, copper salts, gold salts and gallium salts of tungstosilicic acid, tungstophosphoric acid, molybdophosphoric acid, molybdosilicic acid, vanadotungstosilicic acid, vanadotungstophosphoric acid and vanadomolybdophosphoric acid.

10. A process for producing a catalyst for use in production of a lower aliphatic carboxylic acid ester, which comprises, as a step of the process, a step consisting of the method as set forth in any one of claims 1 to 9.

11. A catalyst for use in production of a lower aliphatic carboxylic acid ester, which has been regenerated by the method as set forth in any one of claims 1 to 9.

12. A catalyst for use in production of a lower aliphatic carboxylic acid ester, which has been prepared by the process as set forth in claim 10.

13. A catalyst according to claim 11 or 12, which is a supported catalyst.

14. A catalyst according to any one of claims 11 to 13, which contains a heteropolyacid and/or a heteropolyacid salt.

15. A catalyst according to claim 14, wherein the heteropolyacid is selected from the group consisting of tungstosilicic acid, tungstophosphoric acid, molybdophosphoric acid, molybdosilicic acid, vanadotungstosilicic acid, vanadotungstophosphoric acid, vanadomolybdophosphoric acid, vanadomolybdosilicic acid, molybdotungstosilicic acid and molybdotungstophosphoric acid.

16. A catalyst according to claim 14, wherein the heteropolyacid salt is selected from the group consisting of lithium salts, sodium salts, magnesium salts, barium salts, copper salts, gold salts and gallium salts of tungstosilicic acid, tungstophosphoric acid, molybdophosphoric acid, molybdosilicic acid, vanadotungstosilicic acid, vanadotungstophosphoric acid and vanadomolybdophosphoric acid.

17. A process for producing a lower aliphatic carboxylic acid ester comprising reacting a lower olefin and a lower aliphatic carboxylic acid in a gas phase in the presence of the catalyst as set forth in any one of claims 11 to 16.

18. A process according to claim 17, wherein the reaction of a lower olefin and a lower aliphatic carboxylic acid is carried out in the presence of water.

19. A process according to claim 17 or 18, wherein the lower olefin is selected from the group consisting of ethylene, propylene, n-butene and isobutene.

20. A process according to any one of claims 17 to 19, wherein the lower aliphatic carboxylic acid is selected from the group consisting of formic acid, acetic acid, propionic acid, butyric acid, acrylic acid and a methacrylic acid.

21. A process according to any one of claims 17 to 20, wherein the lower olefin is ethylene and the lower aliphatic carboxylic acid is acetic acid.

22. A process according to any one of claims 17 to 21, which comprises, as a step of the process, a step consisting of the method as set forth in any one of claims 1 to 9.

23. A process for producing a lower aliphatic carboxylic acid ester by reacting a lower olefin and a lower aliphatic carboxylic acid, which comprises the following steps A and B:
Step A
a step for reacting a lower olefin and a lower aliphatic carboxylic acid in a gas phase in a reactor in which a heteropolyacid and/or heteropolyacid salt supported catalyst is filled to produce a lower aliphatic carboxylic acid ester; and
Step B
a step for conducting the method as set forth in any one of claims 1 to 9 while leaving the heteropolyacid and/or heteropolyacid salt supported catalyst filled in the reactor.
